(19) United States
(12) Patent Application Publication
(10) Pub. No.: US 2019/0192021 A1
(43) Pub. Date: Jun. 27, 2019

(54) APPARATUS FOR MEASURING A BIOMAGNETIC FIELD

(71) Applicant: Biomagnetik Park GmbH, Hamburg (DE)

(72) Inventors: Byeongsoo Kim, Hamburg (DE); Bonggun Kim, Hamburg (DE); Malte Ehrlen, Hamburg (DE)

(21) Appl. No.: 16/098,924

(22) PCT Filed: May 8, 2017
(86) PCT No.: PCT/EP2017/060934

§ 371 Date: Nov. 5, 2018

(30) Foreign Application Priority Data
May 9, 2016 (DE) 10 2016 108 524.3

(51) Int. Cl. A61B 5/04; G01R 33/00; G01R 33/035

(52) U.S. Cl. CPC A61B 5/04007; G01R 33/0005; A61B 2562/0223; G01R 33/0094; A61B 2562/046; G01R 33/0354

(57) ABSTRACT

A biomagnetic field measuring apparatus enabling reliable biomagnetic field measurements in clinical practice, having a plurality of magnetic field sensors being arranged in an array in a sensor plane, including a plurality of first magnetic field sensors being designed and configured to measure a first component of the magnetic field, a plurality of second magnetic field sensors being designed and configured to measure a second component of the magnetic field, and a plurality of third magnetic field sensors being designed and configured to measure a third component of the magnetic field, the first, second and third components of the magnetic field being orthogonal to each other. Viewed perpendicular to the sensor plane, the first magnetic field sensors and the second magnetic field sensors are arranged essentially centrally and the third magnetic field sensors are arranged essentially around the first and second magnetic field sensors.

The invention relates to an apparatus for measuring a biomagnetic field.

Apparatus for measuring biomagnetic fields are well known. Examples for such apparatus measuring faint biomagnetic fields, e.g. generated by muscle or nerve tissue, are Magnetocardiographs and Magnetoencephalographs, measuring very weak magnetic fields generated by the electric activity of the heart and the brain, respectively. Biomagnetic field mesuring apparatus are e.g. described in U.S. Pat. No. 5,113,136, 5,644,229, 6,230,037 B1, 6,424 853 B1, 6,842,637 B2, or 7,194,121 B2. Magnetocardiography (MCG) and Magnetoencephalography (MEG) are established non-invasive methods used e.g. for examining subjects for abnormal conditions or diseases of the heart or brain.

There have been several attempts to improve biomagnetic field measuring apparatus, e.g. in using vector magnetocardiographic systems (see. e. g. Thiel et al. 2005, The 304 SQUIDs vector magnetometer system for biomagnetic measurements in the Berlin Magnetically Shielded Room 2, Biomed. Technik (Biomedical Engineering) 50, 169-170; Schnabel et al. 2004, Discrimination of Multiple Sources Using a SQUID Vector Magnetometer, Neurology & Clinical Neurophysiology 2004:67; Jazbinšek et al., 2000, Cardiac multichannel vector MFM and BSPM of front and back thorax, In: Nenonen J, Ilmoniemi R J, Katila T, (ed.), Biomag2000, Proceedings of the 12th Int Conf on Biomagnetism; 2000 Aug. 13-17; Espoo, Finland; Espoo: Helsinki Univ. of Technology; 2001, 583-6; Drung, D., 1995, The PTB 83-SQUID system for biomagnetic applications in a clinic, IEEE Transactions on Applied Superconductivit 5, 2112-2117, doi: 10.1109/77.403000; U.S. Pat. No. 5,644,229).

There is, however, still a need for improving biomagnetic field measuring apparatus, e.g. in view of sensitivity and signal quality.

It is therefore an object of the invention to provide an improved biomagnetic field measuring apparatus, in particular a biomagnetic field measuring apparatus enabling reliable biomagnetic field measurements in clinical practice.

For solving the problem, the invention provides an apparatus for measuring a biomagnetic field comprising a plurality of magnetic field sensors being arranged in an array in a sensor plane, the plurality of magnetic field sensors consisting of a plurality of first magnetic field sensors being designed and configured to measure a first component of the magnetic field, a plurality of second magnetic field sensors being designed and configured to measure a second component of the magnetic field, and a plurality of third magnetic field sensors being designed and configured to measure a third component of the magnetic field, the first, second and third components of the magnetic field being orthogonal to each other, and wherein, viewed from a direction perpendicular to the sensor plane, the first magnetic field sensors and the second magnetic field sensors are arranged essentially centrally and the third magnetic field sensors are arranged essentially around the first and second magnetic field sensors.

It has been found that the sensor arrangement and configuration of the biomagnetic field measuring apparatus of the invention enables sensitive and robust measurements of weak biomagnetic fields, e.g. origination from the heart or brain. The apparatus of the invention is particularly sensitive for small changes in the magnetic field source, e.g. the heart or brain. Thus, the apparatus of the invention is, for example, particularly suitable for the examination of conditions, in which small changes in electric current/magnetic moment are of particular interest, e.g. in the Isolated Left Anterior Descending Coronary Artery Disease (“LAD disease”). The apparatus of the invention also provides for a better inverse solution performance, i.e. a more accurate reconstruction of the electric currents or magnetic moments in the source from the measured magnetic field data. Further, the apparatus of the invention is comparatively insensitive to an offset in relation to the source, e.g. the heart center, making the apparatus of the invention especially suitable for use in a clinical environment.

The term “biomagnetic field” relates to magnetic fields generated by electric currents in cells, tissue or organs, e.g. heart or brain tissue.

The term “magnetic field sensor” as used herein means a sensor being able to measure (bio)magnetic fields. SQUIDs (“superconducting quantum interference devices”, see e.g. Fagaly, R. L., 2006, Superconducting quantum interference device instruments and applications, Rev. Sci. Instrum. 77, 101101, doi: 10.1 063/1.235 4545) are preferred as sensors. The temis “1-axis magnetic field sensor”, “2-axis magnetic field sensor” or “3-axis magnetic field sensor” refer to magnetic field sensors measuring only one, two or three of the three orthogonal components (x, y, z) of the magnetic field, i.e. the. A “3-axis magnetic field sensor” is e.g. a magnetic field sensor measuring the components of the magnetic field in all three dimensions. The term “2-axis magnetic field sensor” encompasses sensors being composed of at least two magnetometers or gradiometers measuring the orthogonal x- and y-, x- and z- or y- and z-components of a magnetic field. Likewise, the term “3-axis magnetic field sensor” encompasses sensors being composed of at least three magnetometers or gradiometers measuring the orthogonal x-, y-, and z-components of a magnetic field.

The term “sensor plane” relates to the plane, in which the sensors, in particular the magnetic field sensing elements, thereof, e.g. detection coils, lie. The term “sensor plane” is not meant to define a plane in a strictly mathematical sense, i.e. a two-dimensional structure, but relates to a two- or three-dimensional (virtual) layer in which the sensors are arranged. In many cases, the sensor plane is essentially parallel to the x-y plane.

The terms “first component”, “second component” or “third component” in relation to a magnetic field refer to the orthogonal components of a magnetic field. Instead, also the terms “x-component” (for e.g. the first component), “y-component” (for e.g. the second component) and “z-component” (for e.g. the third component may be used. The terms refer to the components of any set of orthogonal magnetic field components, without being restricted to a specific meaning of the terms in relation to e.g. a plane or axis of, for example, a human body. In particular, the temis “x-component” and “y-component” preferably refer to the components of the magnetic field in direction of the x- and y-axis, respectively, of a plane (x-y plane) formed by or parallel to a body surface, e.g. the front or back of a human thorax, or the surface of the cranium. The term “z-component” preferably relates in particular to the component in direction of the z-axis, i.e perpendicular to the x-y plane. A reference to an x-axis when measuring magnetic fields of the heart of a human being preferably corresponds to a reference to a right-to-left axis, a reference to an y-axis preferably corresponds to a reference to a head-to-foot axis, and a reference to the z-axis preferably corresponds to a reference to a anteroposterior axis, wherein “right”, “left”, “head”, “foot”, and “anteroposterior” relate to the body of a human being.

The term “source” as used herein means a source of a biomagnetic field or biogmagnetic fields, e.g. the heart or brain. The term encompasses a reference to a reference point source, i.e. to a point taken as the source of all electric and/or magnetic activity of the heart or brain or a heart or brain tissue.

The term “inverse solution” means a solution to the inverse problem. The skilled person is familiar with this problem, and with methods to find an inverse solution, i.e. methods to solve an inverse problem. In the context of the invention the term “inverse solution” refers to methods for reconstructing e.g. the heart or brain activity (i.e. the real electric and/or magnetic activity in the “source space”, the source being the heart or brain, in particular the heart) with data measured in the “sensor space”, i.e. outside the heart or brain.

The terra “inverse solution performance” relates to the quality of an inverse solution for a given source calculated from measured magnetic field data for that source. The “inverse solution performance” can e.g. be evaluated by taking/simulating a given current source, calculating a forward solution for the source and comparing the forward solution with the inverse solution calculated from the measured or simulated magnetic field data of the source.

The term “subject” as used herein refers preferably to a vertebrate, further preferred to a mammal, and most preferred to a human.

The expression according to which a magnetic field sensor is designed and configured to measure a specific component, i.e. the first, second and third component (x-, y- or z-component) of a magnetic field means that the magnetic field sensor is constructed and adapted in a manner that only the respective component of the magnetic field is measured. This does not exclude that a magnetic field sensor is constructed in a manner enabling it to measure one or both of the other components of the magnetic field. Thus, a magnetic field sensor may e.g. be constructed to comprise magnetometers or gradiometers for detecting each of the three magnetic field components, such that the magnetic field component the detector measures can be changed, if desired. The expression according to which a magnetic field sensor is designed and configured to measure e.g. the x-component of a biomagnetic field thus means that a magnetic field sensor may be built to be able to also measure the y and/or z-component of the magnetic field, but is configured to only measure the x-component. Such a configuration may e.g. be established via respective switches or via software.

According to the invention, there are three portions or groups of magnetic field sensors measuring different components of a biomagnetic field and being spatially arranged in a specific manner. A first group of magnetic field sensors measures the first component (x-component) of a biomagnetic field, a second group of magnetic field sensors measures the second component (y-component) of the biomagnetic field, and a third group of magnetic field sensors measures the third component (z-component) of the biomagnetic field. The first, second and third magnetic field sensors are arranged in such a manner, that, viewed from a direction perpendicular to the sensor plane, the first magnetic field sensors and the second magnetic field sensors are arranged essentially centrally and the third magnetic field sensors are arranged essentially around the first and second magnetic field sensors. As already mentioned, the first, second and third magnetic field sensors can all be constructed in a manner that they are also able to measure one or both of the other components of the magnetic field, if configured to do so. According to the invention, the first group of magnetic field sensors is, however, configured to measure the x-component of a biomagnetic field, whereas the second and third group of magnetic magnetic field sensors are configured to measure the y- and z-compent of the biomagnetic field. The plurality of magnetic field sensors are preferably contained in an appropriate housing, e.g. a Dewar vessel as known from the prior art.

In a preferred embodiment the biomagnetic field measuring apparatus of the inveniton the number of first magnetic field sensors, measuring the first component (x-component) of the biomagnetic field, equals the number of second magnetic field sensors, measuring the second component (y-component) of the biomagnetic field.

In a particular preferred embodiment the biomagnetic field measuring apparatus of the inveniton each of the first magnetic field sensors is spatially associated with a second magnetic field sensor, such that both measure the magnetic field components at essentially the same location of a source. In this embodiment of the biomagnetic field measuring apparatus of the inveniton the first and magnetic field sensors form sensor pairs measuring the x- and y-component of the biomagnetic field. It is to be noted here that the sensor pairs may be included in the same housing and may thus form a 2-D-sensor, i.e. a sensor combining two (or more) 1-D-sensors measuring two components of a biomagnetic field, in this case the x- and y-components. As mentioned above, a 3-D-sensor could also be used, i.e. a sensor combining three 1-D-sensors, which are, however, configured to only measure the x- and y-components of the biomagnetic field.

The array of magnetic field sensors can have several forms in terms of its cross-section or area covered when viewed from a direction perpendicular to the sensor plane, e.g. an essentially circular, elliptical, polygonal or rectangular form. In any case, the first and second groups of magnetic field sensors are arranged centrally and the third group magnetic field sensors is arranged in the periphery. In a preferred embodiment of the biomagnetic field measuring apparatus according to the invention (a) the array of magnetic field sensors is, when viewed from a direction perpendicular to the sensor plane, essentially circular, (b) the first magnetic field sensors and the second magnetic field sensors are arranged centrally in an essentially circular region of the array, and (c) the third magnetic field sensors are arranged essentially in a circular region around the first and second magnetic field sensors.

The biomagnetic field measuring apparatus according to the invention may have any suitable number of magnetic field sensors, e.g. 32, 64, 102, or higher number of magnetic field sensors. Preferably, the number of first and second magnetic field sensors is higher than the number of third magnetic field sensors. Preferably, the relation of the number of first and second magnetic field sensors to the number of third magnetic field sensors is about 2-5:1, preferably 2.5-4:1 or 2.5-3:1.

In one embodiment, the biomagnetic field measuring apparatus according to the invention may e.g. comprise 64 magnetic field sensors, wherein 24 first magnetic field sensors and 24 second magnetic field sensors are arranged centrally in an essentially circular portion of the array, and 16 third magnetic field sensors are arranged essentially in a circle region around the circular region containing the first magnetic field sensors and the second magnetic field sensors.

In the following, the invention is described in more detail by way of an example and the attached figures for illustration purposes only.

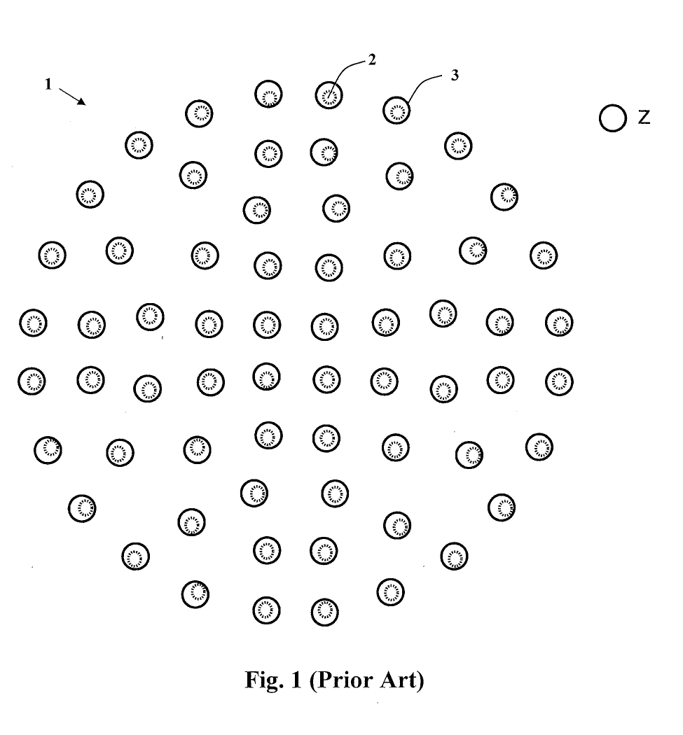

FIG. 1. Schematic illustration of a sensor arrangement according to the prior art.

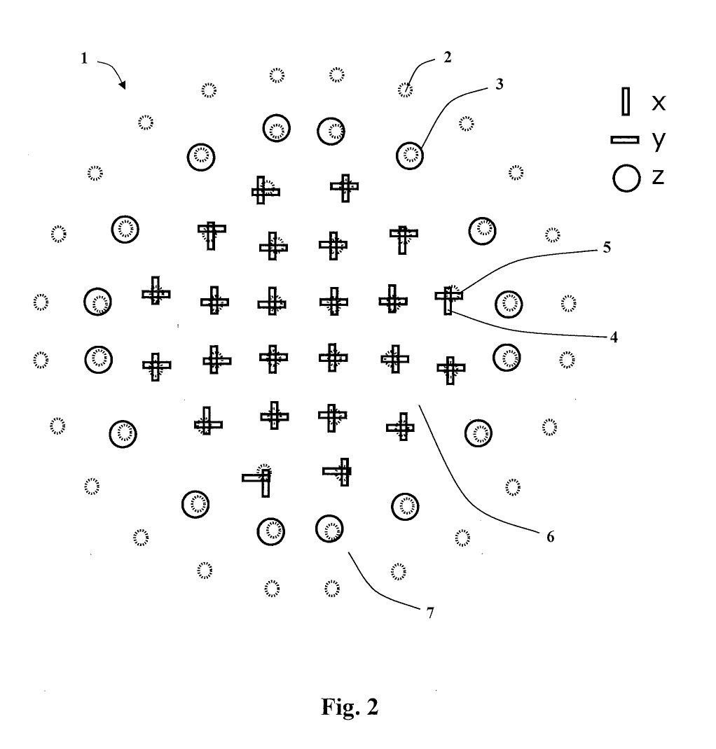

FIG. 2. Schematic illustration of a sensor arrangement according to an embodiment of the invention.

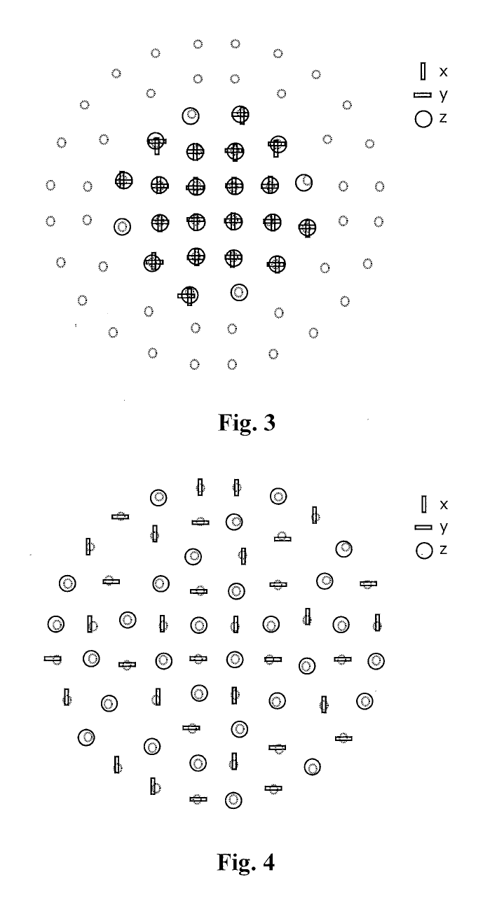

FIGS. 3 and 4. Schematic illustration of examples of comparative sensor arrangements (not according to the invention).

FIG. 1 shows a sensor arrangement according to a prior art 64-channel biomagnetic field measuring apparatus. Circles with dotted outlines denoted with the reference numeral 2 represent measuring points on a magnetic source, here the heart. Magnetic field sensors 3 measuring the z-component of the biomagnetic field generated by the heart at the measuring points are arranged in an essentially circular array 1. All of the 64 magnetic field sensors 3 of the prior art apparatus are of one type, i.e. a type measuring only the z-component of the biomagnetic field.

FIG. 2 shows a sensor arrangement according to an embodiment of the invention for a 64-channel biomagnetic field measuring apparatus, in this case an MCG. For comparison, the 64 measuring points 2 of the prior art apparatus of FIG. 1 are also depicted here. 24 first magnetic field sensors 4 and 24 second magnetic field sensors 5 are arranged in an essentially circular region 6 of the array 1. Each of the 24 first magnetic field sensors 4 is associated with a corresponding second magnetic field sensor 5, such that sensor pairs thus formed measure the x- and y-components of the biomagnetic field at the same measuring point. 16 third magnetic field sensors 3 measuring the z-component of the biomagnetic field are arranged in an essentially circular or annular region 7 around or in the periphery of the first and second magnetic field sensors 4, 5.

FIGS. 3 and 4 show two other sensor configurations (not according to the invention) used for the purpose of comparison. In FIG. 3 a sensor configuration is shown in which all sensors are distributed over the cross-section of the central circular region 6. The arrangement is composed of 4 sensors measuring only the z-component of the magnetic field at the corners of a quadrangular area within the central circular region 6, and 3×20 sensors measuring the x-, y- and z-components at corresponding 20 measuring points, respectively. FIG. 4 depicts an arrangement, in which each of the 64 measuring points 2 is associated with one of 64 magnetic field sensos, 18 of the 64 sensors measuring the x-component of the magnetic field, 17 sensors measuring the y-component of the magnetic field and 29 sensors measuring the z-component of the magnetic field.

An MCG having a sensor configuration according to the embodiment of the invention shown in FIG. 2 was compared with MCGs set-up with a prior art sensor configuration according to the one depicted in FIG. 1 and with MCGs set-up with the sensor configurations of FIGS. 3 and 4, respectively. Small changes of the current dipole pattern on the frontal area of the heart were simulated. The prior art 64-channel MCG calculated 298 dipoles on the heart.

The results showed that the sensor configuration of the invention (FIG. 2) and the configuration according to FIG. 3 are superior to the configurations according to the prior art (FIG. 1) and according to FIG. 4 in order to explain the small changes.

Further, the inverse solution performance of the different sensor arrangements was evaluated. A forward model was calculated from a given source and the inverse solution was calculated from the measured magnetic field data. It could be shown that, by comparing the original source and the inverse solution, that the sensor configuration according to the invention (FIG. 2) and the sensor configuration according to FIG. 3 have a better inverse solution performance than the prior art sensor configuration and the sensor configuration according to FIG. 4.

The robustness of the compared sensor configurations in view of an offset from the heart center was evaluated. For this purpose a position offset in x-direction (right hand to left hand) was simulated. It could be shown that the prior art sensor configuration has a bigger anle error than the sensor configuration according to the invention and the sensor configuration according to FIG. 4.

In summary, it was shown that an MCG having a sensor configuration of the invention according to FIG. 2 is superior in view of sensitivity and robustness compared to the prior art.

1. Apparatus for measuring a biomagnetic field comprising a plurality of magnetic field sensors (3, 4, 5) being arranged in an array (1) in a sensor plane, the plurality of magnetic field sensors (3, 4, 5) consisting of a plurality of first magnetic field sensors (4) being designed and configured to measure a first component of the magnetic field, a plurality of second magnetic field sensors (5) being designed and configured to measure a second component of the magnetic field, and a plurality of third magnetic field sensors (3) being designed and configured to measure a third component of the magnetic field, the first, second and third components of the magnetic field being orthogonal to each other, and wherein, viewed from a direction perpendicular to the sensor plane, the first magnetic field sensors (4) and the second magnetic field sensors (5) are arranged essentially centrally and the third magnetic field sensors (3) are arranged essentially around the first and second magnetic field sensors (4, 5).

2. The biomagnetic field measuring apparatus according to claim 1, wherein the number of first magnetic field sensors (4) equals the number of second magnetic field sensors (5).

3. The biomagnetic field measuring apparatus according to claim 2, wherein each of the first magnetic field sensors (4) is spatially associated with a second magnetic field sensor (5), such that both measure the magnetic field components at essentially the same location of a source.

4. The biomagnetic field measuring apparatus according to one of the preceding claims claim 1, wherein the array (1) of magnetic field sensors (3, 4, 5) has, viewed from a direction perpendicular to the sensor plane, an essentially circular, elliptical, rectangular or polygonal shape.

5. The biomagnetic field measuring apparatus according to claim 4, wherein (a) the array (1) of magnetic field sensors (3, 4, 5) is, viewed from a direction perpendicular to the sensor plane, essentially circular, (b) the first magnetic field sensors (4) and the second magnetic field sensors (5) are arranged centrally in an essentially circular region (6) of the array, and (c) the third magnetic field sensors (3) are arranged essentially in a circular region (7) around the first and second magnetic field sensors (4, 5).

6. The biomagnetic field measuring apparatus according to claim 5, comprising 64 magnetic field sensors (3, 4, 5), wherein 24 first magnetic field sensors (4) and 24 second magnetic field sensors (5) are arranged centrally in an essentially circular portion of the array, and 16 third magnetic field sensors (3) are arranged essentially in a circular region (7) around the circular region (6) containing the first magnetic field sensors (4) and the second magnetic field sensors (5).

7. The biomagnetic field measuring apparatus according to one of the preceding claims claim 1, wherein the biomagnetic field measuring apparatus is a magnetocardiograph.

8. Apparatus for measuring a biomagnetic field comprising a plurality of magnetic field sensors (3, 4, 5) being arranged in an array (1) in a sensor plane, the plurality of magnetic field sensors (3, 4, 5) comprising a plurality of first magnetic field sensors (4) being designed and configured to measure a first component of the magnetic field, a plurality of second magnetic field sensors (5) being designed and configured to measure a second component of the magnetic field, and a plurality of third magnetic field sensors (3) being designed and configured to measure a third component of the magnetic field, the first, second and third components of the magnetic field being orthogonal to each other, and wherein, viewed from a direction perpendicular to the sensor plane, the first magnetic field sensors (4) and the second magnetic field sensors (5) are arranged essentially centrally and the third magnetic field sensors (3) are arranged essentially around the first and second magnetic field sensors (4, 5).
